(19) United States
(12) Patent Application Publication
(10) Pub. No.: US 2023/0125693 A1
(43) Pub. Date: Apr. 27, 2023

(54) BREATHING CIRCUIT-TO-LARYNGOSCOPE ADAPTER

(71) Applicant: Lisa Monique Staples, South Euclid, OH (US)

(72) Inventor: Lisa Monique Staples, South Euclid, OH (US)

(21) Appl. No.: 17/451,874

(22) Filed: Oct. 22, 2021

(51) Int. Cl. A61M 39/10

(52) U.S. Cl. CPC A61M 39/10; A61M 2039/1077; A61B 1/2673

(57) ABSTRACT

A unitary adapter includes a body having a small-bore connector formed at a first end and an opening formed at a second having a larger circumference than the small-bore connector. The small-bore connector couples the body and a operating laryngoscope. The larger opening end couples the body and a breathing circuit. The unitary adapter connects the breathing circuit to the operating laryngoscope.

### CROSS-REFERENCE TO RELATED APPLICATION

N/A

### BACKGROUND OF THE INVENTION

The present invention relates to a surgical breathing circuit and, more particularly, to a breathing circuit-to-laryngoscope adapter.

During some surgical procedures, it is necessary for the anesthesiologist to ventilate the subject. A breathing circuit has a universal connection size and a laryngoscope has a universal connection with a different size, as does its attachment piece. As there currently are no commercially available devices for attachment to both the laryngoscope and the breathing circuit, the anesthesiologist must pass an endotracheal tube through the laryngoscope and attach it to the breathing circuit. This arrangement takes up space in the surgical field and interferes with necessary surgical procedures.

As can be seen, there is a need for an adapter device to connect the breathing circuit to the laryngoscope.

### SUMMARY OF THE INVENTION

In one aspect of the present invention, a unitary adapter for coupling a breathing circuit to an operating laryngoscope is provided, comprising a body having a small-bore connector formed at a first end configured to couple the body and the operating laryngoscope; and an opening formed at a second end configured to couple the body and the breathing circuit, said opening having a larger circumference than the small-bore connector.

These and other features, aspects and advantages of the present invention will become better understood with reference to the following drawings, description, and claims.

### BRIEF DESCRIPTION OF THE DRAWINGS

FIG. 1 is a perspective view of a laryngoscope-breathing circuit adapter according to an embodiment of the present invention;

FIG. 2 is an exploded view thereof;

FIG. 3 is a front elevation view thereof; and

FIG. 4 is a sectional view thereof, taken along line 4-4 on FIG. 3.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, one embodiment of the present invention is a unitary adapter for connecting a breathing circuit to an operating laryngoscope or its attachment piece, such as a lateral port.

The adapter enables an anesthesiologist to ventilate without interfering in surgical field. Spontaneous ventilation is possible when the adapter is added to the side of a laryngoscope or its attachment. The adapter opens up the surgical field by allowing the breathing circuit to connect to a laryngoscope outside of the field.

The adapter may include a body with a first end, a second end, and a flange therebetween. The body may have a flange with a rounded rectangle shape.

A first end of the adapter is a small-bore connector such as a Luer-Lok® fitting configured to attach to a side attachment piece of an operating laryngoscope or to the laryngoscope directly. The small-bore connector may be approximately 2 inches long with an approximately ½-inch circumference. In some embodiments, the Luer-Lok® connector may be replaced by another small-bore connector configured to couple the body and the laryngoscope and/or its side attachment piece.

A second end of the adapter has a larger opening than the first end, i.e., the opening has a larger circumference than that of the small-bore connector. The larger opening of the second end is configured to couple the body and a breathing circuit and is approximately 1½″ in circumference.

The inventive adapter may be formed as one piece using a mold. The molded piece is open on opposite ends with a central passage between the ends so that air or another gas passes through. The material of manufacture is not particularly limited and may be, for example, a hard plastic.

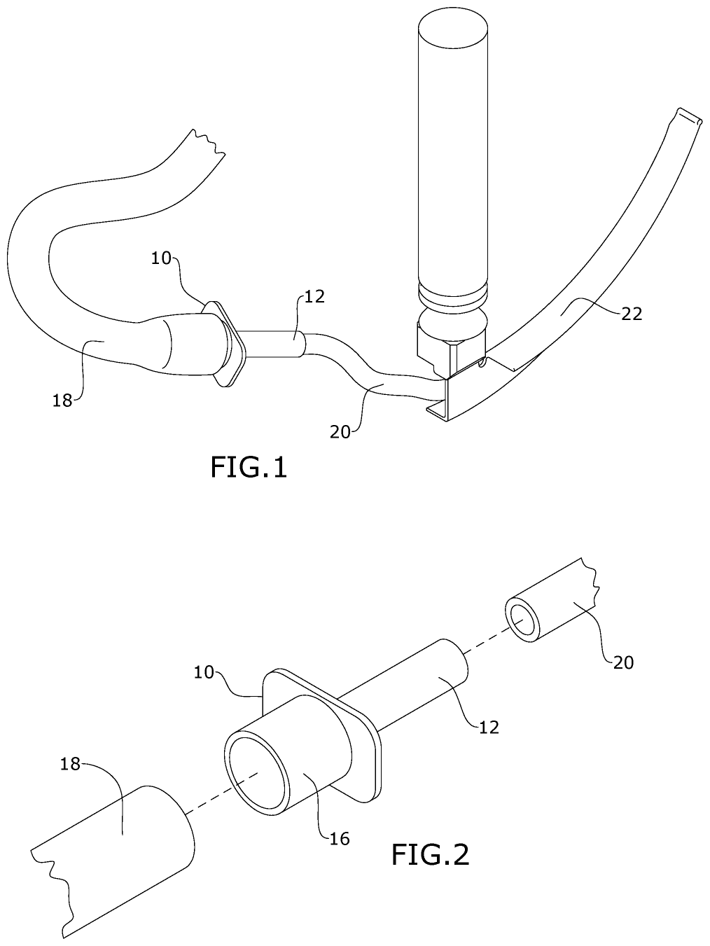

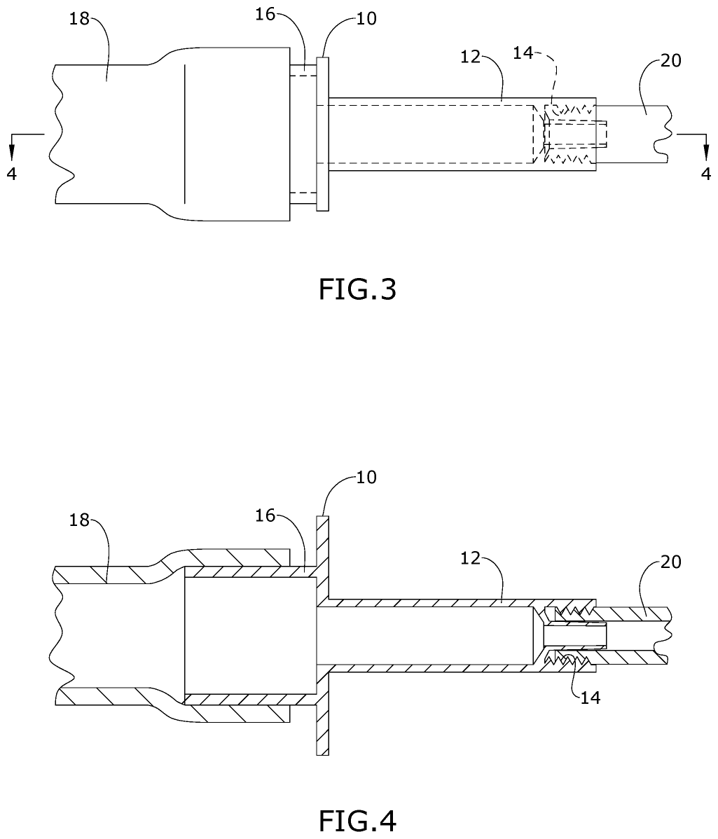

Referring to FIGS. 1 through 4, FIG. 1 shows an adapter 10 in use, connecting a breathing circuit 18 to laryngoscope attachment piece 20 and thereby to a laryngoscope 22. As better seen in FIG. 2, the adapter 10 has a small end 12 for connection to the laryngoscope attachment piece 20. Opposite the small end 12, the adapter has a large end 16 for connection to the breathing circuit 18. As shown in FIGS. 3 and 4, the small end 12 are shown with a Luer-Lok® connector 14 that couples with the laryngoscope attachment piece 20.

It should be understood, of course, that the foregoing relates to exemplary embodiments of the invention and that modifications may be made without departing from the spirit and scope of the invention as set forth in the following claims.

What is claimed is:

1. A unitary adapter for coupling a breathing circuit to an operating laryngoscope, comprising:
a body having
a small-bore connector formed at a first end configured to couple the body and the operating laryngoscope; and
an opening formed at a second end configured to couple the body and the breathing circuit, said opening having a larger circumference than the small-bore connector.

2. The unitary adapter of claim 1, wherein the small-bore connector is configured to couple the body and an attachment port on a side of the operating laryngoscope.

3. The unitary adapter of claim 1, wherein the small-bore connector is configured to couple the body and an end of the operating laryngoscope.

4. The unitary adapter of claim 1, wherein the body has a rounded rectangle-shaped flange between the first end and the second end.
